(19) United States
(12) Patent Application Publication
(10) Pub. No.: US 2016/0201085 A1
(43) Pub. Date: Jul. 14, 2016

(54) Enhancing Efficiency of Retroviral Transduction of Host Cells

(71) Applicants: Bruce TORBETT, Encinitas, CA (US); Cathy X. WANG, La Jolla, CA (US); THE SCRIPPS RESEARCH INSTITUTE, La Jolla, CA (US)

(72) Inventors: Bruce E. Torbett, Encinitas, CA (US); Cathy X. Wang, La Jolla, CA (US)

(21) Appl. No.: 14/913,579

(22) PCT Filed: Aug. 22, 2014
(86) PCT No.: PCT/US14/52258

§ 371 Date: Feb. 22, 2016

Related U.S. Application Data
(60) Provisional application No. 61/869,172, filed on Aug. 23, 2013.

(51) Int. Cl. C12N 15/85; C12N 7/00; C12N 15/86

(52) U.S. Cl. CPC C12N 15/85; C12N 15/86; C12N 7/00; C12N 2740/15043

(57) ABSTRACT

The present invention provides methods for enhancing transduction efficiency of a viral vector into a host cell such as an unstimulated stem cell. The methods involve transducing the host cell with the vector in the presence of an SAMHD1 inhibitor (e.g., a Vpx protein), and an inhibitor of mTOR complexes (e.g., rapamycin or analog compound thereof). Also provided in the invention are kits or pharmaceutical combinations for delivering a therapeutic agent into a target cell with enhanced targeting frequency and payload delivery. The kits or pharmaceutical combinations typically contain a viral vector encoding the therapeutic agent, an SAMHD1 inhibitor or a polynucleotide encoding the SAMHD1 inhibitor, and an inhibitor of mTOR complexes.

### CROSS-REFERENCE TO RELATED APPLICATIONS

The subject patent application claims the benefit of priority to U.S. Provisional Patent Application No. 61/869,172 (filed Aug. 23, 2013). The full disclosure of the priority application is incorporated herein by reference in its entirety and for all purposes.

### BACKGROUND OF THE INVENTION

Viruses are highly efficient at nucleic acid delivery to specific cell types, while often avoiding detection by the infected host immune system. These features make certain viruses attractive candidates as gene-delivery vehicles for use in gene therapies. Retroviral vectors are the most commonly used gene delivery vehicles. The retroviral genome becomes integrated into host chromosomal DNA, ensuring its long-term persistence and stable transmission to all future progeny of the transduced cell and making retroviral vector suitable for permanent genetic modification. Retroviral based vectors can be manufactured in large quantities, which allow their standardization and use in pharmaceutical preparations.

Hematopoietic stem cells (HSCs), long-lived precursors to the entire hematopoietic system, are intrinsically refractory to HIV-1 replication. Human CD34⁺ hematopoietic stem and progenitor cells can be infected in vitro at low levels, but occurrence of in vivo infection remains controversial. Similarly, they are refractory to transduction by HIV-1 based lentiviral vectors, greatly hampering the efficacy of HSC gene therapy. NOD/SCID-repopulating cells—experimentally defined as truly primitive HSCs—show only low levels of lentiviral-mediated gene marking, which cannot be overcome even by extremely high vector-to-cell ratios. The block is thought to occur post-entry, as primary HSCs express HIV-1 receptors, and lentiviral vectors are commonly pseudotyped with the vesicular stomatitis virus glycoprotein (VSV-G) to allow for ubiquitous tropism.

There is a need in the art for conditions that promote efficient transduction of retroviral vectors, esp. lentiviral vectors such as HIV based vectors, into various host cells (e.g., stem cells and other hematopoietic cells, such as T cells) for gene transfer in a rapid manner without cytokine activation. The present invention addresses this and other needs.

### SUMMARY OF THE INVENTION

In one aspect, the invention provides methods for enhancing transduction efficiency of a viral vector into a host cell. The methods entail transducing the host cell with the viral vector in the presence of (1) an mTOR inhibitor compound and (2) an inhibitor of SAM domain and HD domain-containing protein 1 (SAMHD1). In some of the methods, the SAMHD1 inhibitor is packaged along with the viral vector into a virion prior to transducing the host cell. In some embodiments, the host cell is not pre-stimulated with cytokine prior to transduction of the vector. Some of these methods are directed to transducing an unstimulated stem cell or a resting T cell, e.g., a hematopoietic stem cell (HSC). In various embodiments, the host cell is present in vivo, e.g., in a human or non-human subject.

In some embodiments, the viral vector to be transduced is a lentiviral vector. For example, the viral vector can be a HIV-1 based vector. In some embodiments of the invention, the SAMHD1 inhibitor is accessory protein viral protein X (Vpx) or viral protein R (Vpr). The accessory protein Vpx to be used in the invention can be encoded, e.g., by HIV-2, SIV_{SM}, or SIV_{MAC}. The accessory protein Vpr suitable for the invention can be encoded by, e.g., SIVmus and SIVdeb. In some embodiments of the invention, the mTOR inhibitor to be employed can be a molecule that inhibits or antagonizes mTOR Complex 1 (mTORC1) and/or mTOR Complex 2 (mTORC2). In some embodiments, the employed mTOR inhibitor is rapamycin or analog compound thereof.

In practicing some embodiments of the invention, the viral vector can be transduced into the stem cell at a multiplicity of infection (MOI) of, e.g., 5, 10, 25, 50 or 100. In various embodiments, the mTOR inhibitor compound can be present during the entire transduction process or at specific intervals. In some embodiments, the viral vector can encode a therapeutic agent. In some embodiments, the employed viral vector is a non-integrating lentiviral vector.

In another aspect, the invention provides kits or pharmaceutical combinations for delivering a therapeutic agent into a target cell with enhanced targeting frequency and payload delivery. In some embodiments, the kits contain (a) a viral vector encoding the therapeutic agent, (b) an inhibitor of mTOR complexes, and (c) an SAMHD1 inhibitor or a polynucleotide encoding the SAMHD1 inhibitor. In some embodiments, the mTOR inhibitor is rapamycin or an analog thereof, and the SAMHD1 inhibitor is a Vpx or Vpr protein or functional fragment thereof Some embodiments further contain reagents for packaging the SAMHD1 inhibitor with the viral vector into a virion. In some kits, the viral vector is a lentiviral vector.

A further understanding of the nature and advantages of the present invention may be realized by reference to the remaining portions of the specification and claims.

### DESCRIPTION OF THE DRAWINGS

FIG. 1 shows that efficient lentiviral vector transduction of non-cytokine stimulated human CD34+ cells requires both rapamycin treatment and the presence of Vpx in the virion. Left panel shows the percentages of hematopoietic cells marked with GFP after transduction with lentiviral vectors without or with Vpx in the virion where non-cytokine treated CD34+ HSCs were not treated (filled box) or treated with 10 ug/ml (open circle) or 20 ug/ml (x) of rapamycin. The panel on the right presents the Mean Fluorescence Intensity (MFI) for the same treatment groups presented in the Left Panel. The MFI is a surrogate for the number of integrated vectors per cell. Total treatment time was 12 hours.

FIG. 2 shows that the rapamycin also enhances lentiviral vector transduction of resting, unstimulated CD4+ T cells.

### DETAILED DESCRIPTION OF THE INVENTION

### I. Overview

The present invention provides for effective means to enhance transduction of retroviral vector or virions (e.g., lentiviruses such as HIV) into various host cells, including unstimulated stem cells or resting T cells. As exemplified herein, HIV-1 transduction into freshly isolated CD34+ cells within 12 hours in the absence of cytokine stimulation was greatly enhanced via methods described herein. Some embodiments of the invention entail transducing the host cells (e.g., CD34+ cells) in the presence of an mTOR inhibitor (e.g., rapamycin or Torin) and also an inhibitor of the early-acting restriction factor, SAMHD1. SAMHD1 is a deoxynucleoside triphosphohydrolase that cleaves dNTPs to produce deoxynucleosides and triphosphates and also exhibits 3′ to 5′ exonuclease acitivity. In some embodiments, the SAMHD1 inhibitor is the HIV-2 accessory protein Vpx which can abolish the activity of SAMHD1. In some embodiments, the Vpx protein is packaged within the HIV-1 virion during lentiviral virion packaging. As exemplified herein, the combination of an mTOR inhibitor and an SAMHD1 inhibitor produces a synergistic effect in enhancing viral transduction into the host cells.

There are various advantages associated with methods of the present invention. For example, the methods allow for effective transduction of nonstimulated stem cells or resting T cells. In some embodiments, there is no requirement for cytokine prestimulation or cell proliferation. The transduced cells, having not been exposed to cytokines, can remain pluripotent. In addition, the viral transduction period is short, typically 12 hours or less for HSC. On the other hand, current protocols known in the art require up to 2-3 days in tissue culture to effectively transduce HSCs, which could reduce HSC pluripotency. Further, methods of the invention are also suitable for efficient transduction of other host cells beside stem cells, e.g., resting T cells. Methods of the invention are superior to prior art methods for direct injections (e.g., in vivo gene delivery) into bone marrow, and other hematopoietic or lymphoid containing organs.

### II. Definition

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “analog” is used herein to refer to a molecule that structurally resembles a reference molecule but which has been modified in a targeted and controlled manner, by replacing a specific substituent of the reference molecule with an alternate substituent. Compared to the reference molecule (e.g., rapamycin), an analog can exhibit the same, similar, or improved utility. Methods for synthesizing and screening candidate analog compounds of a reference molecule to identify analogs having altered or improved traits (e.g., a rapamycin analog compound with enhanced inhibitory activity than rapamycin on lymphocyte response to IL-2) are well known in the art.

The term “contacting” has its normal meaning and refers to combining two or more agents (e.g., two compounds or a compound and a cell) or combining agents and cells.

Contacting can occur in vitro, e.g., mixing a compound and a cultured cell in a test tube or other container. It can also occur in vivo (contacting a compound with a cell within a subject) or ex vivo (contacting the cell with compound outside the body of a subject and followed by introducing the treated cell back into the subject).

Host cell restriction refers to resistance or defense of cells against viral infections. Mammalian cells can resist viral infections by a variety of mechanisms. Viruses must overcome host cell restrictions to successfully reproduce their genetic material.

Retroviruses are enveloped viruses that belong to the viral family Retroviridae. The virus itself stores its nucleic acid, in the form of a +mRNA (including the 5′-cap and 3′-PolyA inside the virion) genome and serves as a means of delivery of that genome into host cells it targets as an obligate parasite, and constitutes the infection. Once in a host's cell, the virus replicates by using a viral reverse transcriptase enzyme to transcribe its RNA into DNA. The DNA is then integrated into the host's genome by an integrase enzyme. The retroviral DNA replicates as part of the host genome, and is referred to as a provirus. Retroviruses include the genus of Alpharetrovirus (e.g., avian leukosis virus), the genus of Betaretrovirus; (e.g., mouse mammary tumor virus), the genus of Gammaretrovirus (e.g., murine leukemia virus or MLV), the genus of Deltaretrovirus (e.g., bovine leukemia virus and human T-lymphotropic virus), the genus of Epsilonretrovirus (e.g., Walleye dermal sarcoma virus), and the genus of Lentivirus.

Lentivirus is a genus of viruses of the Retroviridae family, characterized by a long incubation period. Lentiviruses can deliver a significant amount of genetic information into the DNA of the host cell, so they are one of the most efficient methods of a gene delivery vector. Examples of lentiviruses include human immunodeficiency viruses (HIV-1 and HIV-2), simian immunodeficiency virus (SIV), and feline immunodeficiency virus (FIV). Additional examples include BLV, EIAV and CEV.

mTOR, or the “mammalian target of rapamycin,” is a protein that in humans is encoded by the FRAP1 gene. mTOR is a serine/threonine protein kinase that regulates cell growth, cell proliferation, cell motility, cell survival, protein synthesis, and transcription. mTOR, which belongs to the phosphatidylinositol 3-kinase-related kinase protein family, is the catalytic subunit of two molecular complexes: mTORC1 and mTORC2.

mTOR Complex 1 (mTORC1) is composed of mTOR, regulatory-associated protein of mTOR (Raptor), mammalian lethal with SEC13 protein 8 (MLST8) and partners PRAS40 and DEPTOR. This complex is characterized by the classic features of mTOR by functioning as a nutrient/energy/redox sensor and controlling protein synthesis. The activity of this complex is stimulated by insulin, growth factors, serum, phosphatidic acid, amino acids (particularly leucine), and oxidative stress. mTOR Complex 2 (mTORC2)is composed of mTOR, rapamycin-insensitive companion of mTOR (RICTOR), GβL, and mammalian stress-activated protein kinase interacting protein 1 (mSIN1). mTORC2 has been shown to function as an important regulator of the cytoskeleton through its stimulation of F-actin stress fibers, paxillin, RhoA, Rac1, Cdc42, and protein kinase C α (PKCα). mTORC2 also appears to possess the activity of a previously elusive protein known as “PDK2”. mTORC2 phosphorylates the serine/threonine protein kinase Akt/PKB at a serine residue S473.

The term “mutagenesis” or “mutagenizing” refers to a process of introducing changes (mutations) to the base pair sequence of a coding polynucleotide sequence and consequential changes to its encoded polypeptide. Unless otherwise noted, the term as used herein refers to mutations artificially introduced to the molecules as opposed to naturally occurring mutations caused by, e.g., copying errors during cell division or that occurring during processes such as meiosis or hypermutation. Mutagenesis can be achieved by a number of means, e.g., by exposure to ultraviolet or ionizing radiation, chemical mutagens, or viruses. It can also be realized by recombinant techniques such as site-specific mutagenesis, restriction digestion and religation, error-prone PCR, polynucleotide shuffling and etc. For a given polynucleotide encoding a target polypeptide, mutagenesis can result in mutants or variants that contain various types of mutations, e.g., point mutations (e.g., silent mutations, missense mutations and nonsense mutations), insertions, or deletions.

The term “operably linked” when referring to a nucleic acid, means a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

The term “polynucleotide” or “nucleic acid” as used herein refers to a polymeric form of nucleotides of any length, either ribonucleotides or deoxyribonucleotides, that comprise purine and pyrimidine bases, or other natural, chemically or biochemically modified, non-natural, or derivatized nucleotide bases. Polynucleotides of the embodiments of the invention include sequences of deoxyribopolynucleotide (DNA), ribopolynucleotide (RNA), or DNA copies of ribopolynucleotide (cDNA) which may be isolated from natural sources, recombinantly produced, or artificially synthesized. A further example of a polynucleotide is polyamide polynucleotide (PNA). The polynucleotides and nucleic acids may exist as single-stranded or double-stranded. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The polymers made of nucleotides such as nucleic acids, polynucleotides and polynucleotides may also be referred to herein as nucleotide polymers.

Polypeptides are polymer chains comprised of amino acid residue monomers which are joined together through amide bonds (peptide bonds). The amino acids may be the L-optical isomer or the D-optical isomer. In general, polypeptides refer to long polymers of amino acid residues, e.g., those consisting of at least more than 10, 20, 50, 100, 200, 500, or more amino acid residue monomers. However, unless otherwise noted, the term polypeptide as used herein also encompass short peptides which typically contain two or more amino acid monomers, but usually not more than 10, 15, or 20 amino acid monomers.

Proteins are long polymers of amino acids linked via peptide bonds and which may be composed of two or more polypeptide chains. More specifically, the term “protein” refers to a molecule composed of one or more chains of amino acids in a specific order; for example, the order as determined by the base sequence of nucleotides in the gene coding for the protein. Proteins are essential for the structure, function, and regulation of the body's cells, tissues, and organs, and each protein has unique functions. Examples are hormones, enzymes, and antibodies. In some embodiments, the terms polypeptide and protein may be used interchangeably.

Stem cells are biological cells found in all multicellular organisms, and can divide (through mitosis) and differentiate into diverse specialized cell types and can self-renew to produce more stem cells. In mammals, there are two broad types of stem cells: embryonic stem cells, which are isolated from the inner cell mass of blastocysts, and adult stem cells, which are found in various tissues. In adult organisms, stem cells and progenitor cells act as a repair system for the body, replenishing adult tissues. In a developing embryo, stem cells can differentiate into all the specialized cells (these are called pluripotent cells), but also maintain the normal turnover of regenerative organs, such as blood, skin, or intestinal tissues. There are three accessible sources of autologous adult stem cells in humans: bone marrow, adipose tissue (lipid cells) and blood. Stem cells can also be taken from umbilical cord blood just after birth.

Hematopoietic stem cells (HSCs) are a heterogeneous population of multipotent stem cells that can give rise to all the blood cell types from the myeloid (monocytes and macrophages, neutrophils, basophils, eosinophils, erythrocytes, megakaryocytes/platelets, dendritic cells), and lymphoid lineages (T-cells, B-cells, NK-cells). These cells are found in the bone marrow of adults; within femurs, pelvis, ribs, sternum, and other bones. The cells can usually be obtained directly from the iliac crest part of the pelvic bone, using a special needle and a syringe. They are also collected from the peripheral blood following pre-treatment with cytokines, such as G-CSF (granulocyte colony-stimulating factors) or other reagents that induce cells to be released from the bone marrow compartment. Other sources for clinical and scientific use include umbilical cord blood, as well as peripheral blood.

A cell has been “transformed” or “transfected” by exogenous or heterologous polynucleotide when such polynucleotide has been introduced inside the cell. The transforming polynucleotide may or may not be integrated (covalently linked) into the genome of the cell. In prokaryotes, yeast, and mammalian cells for example, the transforming polynucleotide may be maintained on an episomal element such as a plasmid. With respect to eukaryotic cells, a stably transformed cell is one in which the transforming polynucleotide has become integrated into a chromosome so that it is inherited by daughter cells through chromosome replication. This stability is demonstrated by the ability of the eukaryotic cell to establish cell lines or clones comprised of a population of daughter cells containing the transforming polynucleotide. A “clone” is a population of cells derived from a single cell or common ancestor by mitosis. A “cell line” is a clone of a primary cell that is capable of stable growth in vitro for many generations.

Sterile alpha motif domain and HD domain-containing protein 1 (SAMHD1) is a protein that in humans is encoded by the SAMHD1 gene. SAMHD1 is a cellular enzyme, responsible for blocking replication of HIV in dendritic cells, macrophages and monocytes. It is an enzyme that exhibits phosphohydrolase activity, converting nucleotide triphosphates to a nucleoside and triphosphate. In doing so, SAMHD1 depletes the pool of nucleotides available to a reverse transcriptase for viral cDNA synthesis and thus prevents viral replication. SAMHD1 also has nuclease activity.

SAMHD1 was identified as the cellular protein responsible of the reverse transcription block to HIV-1 infection observed in myeloid cells as well as in quiescent CD4+ T cells. SAMHD1 inhibits HIV-1 infection in myeloid cells by limiting the intracellular pool of dNTPs. The dNTP triphosphohydrolase activity of SAMHD1 has been proposed to reduce the intracellular dNTP level, restricting HIV-1 replication and preventing activation of the immune system, a nuclease activity against single-stranded (ss)DNAs and RNAs, as well as against RNA in DNA/RNA hybrids. Retroviral restriction ability of SAMHD1 requires phosphorylation, for this purpose SAMHD1 associates with the cyclin A2/CDK1 complex that mediates its phosphorylation at threonine 592.

Viral protein X (Vpx) is an accessory protein encoded by human immunodeficiency virus and some simian immunodeficiency viruses (SIVs). Vpx promotes viral infection of host cells, e.g., macrophages and DCs, by targeting and counteracting SAMHD1-mediated restriction. Vpx recruits SAMHD1 to a cullin4A-RING E3 ubiquitin ligase (CRL4), which targets the enzyme for proteasomal degradation.

A “variant” of a reference molecule (e.g., rapamycin) refers to a molecule which has a structure that is derived from or similar to that of the reference molecule. Typically, the variant is obtained by modification of the reference molecule in a controlled or random manner. As detailed herein, methods for modifying a reference molecule to obtain functional derivative compounds that have similar or improved properties relative to that of the reference molecule are well known in the art.

A “vector” is a replicon, such as plasmid, phage or cosmid, to which another polynucleotide segment may be attached so as to bring about the replication of the attached segment. Vectors capable of directing the expression of genes encoding for one or more polypeptides are referred to as “expression vectors”.

A retrovirus (e.g., a lentivirus) based vector or retroviral vector means that genome of the vector comprises components from the virus as a backbone. The viral particle generated from the vector as a whole contains essential vector components compatible with the RNA genome, including reverse transcription and integration systems. Usually these will include the gag and pol proteins derived from the virus. If the vector is derived from a lentivirus, the viral particles are capable of infecting and transducing non-dividing cells. Recombinant retroviral particles are able to deliver a selected exogenous gene or polynucleotide sequence such as therapeutically active genes, to the genome of a target cell.

### III. SAMHD1 Inhibitors for Enhancing Viral Transduction

Various compositions and methods of the invention can employ an inhibitor of restriction factor SAMHD1 in combination with an mTOR inhibitor compound (e.g., rapamycin) to enhance retroviral transduction. The inhibitors can be applied to the target host cells prior to, concurrently with or subsequent to contacting the viral vector or virion with the cells. When the inhibitors (esp. the SAMHD1 inhibitor) are applied to the cells concurrently with contacting the viral vector or virion with the cells, they can be present either independent of the virion or as part of the virion. In the latter case, the inhibitors (e.g., the SAMHD1 inhibitor) can be attached to the virion via, e.g., chemical conjugation or recombinant expression. For example, as detailed herein, a viral protein X (Vpx) or viral protein R (Vpr) can be expressed on the virion by packaging along with a retroviral vector into the virion.

SAMHD1 inhibitors of any chemical classes can be utilized in the invention. These include, e.g., SAMHD1-inhibiting proteins or polypeptides such as Vpx or Vpr proteins and their fragments. Suitable SAMHD1 inhibitors also include small molecule organic compounds that can inhibit or antagonize one or more cellular or biological activities of SAMHD1, e.g., its enzymatic activities. Such inhibitors can be readily obtained by screening of a library of candidate compounds using standard protocols, e.g., combinatory library screening methods. In some embodiments, the employed SAMHD1 inhibitor is accessory protein Vpx or Vpr described herein, or functional analog or fragment thereof. These include polypeptides or peptides derived from a Vpx or Vpr protein that possess the inhibitor function.

The accessory protein Vpx is critical for the ability of primate lentiviruses to efficiently infect monocytes, dendritic cells, and mature macrophages. Vpx allows primate lentiviruses to infect key immunomodulatory cells types by targeting the restriction factor SAMHD1 for degradation. SAMHD1 is a deoxynucleotide triphosphohydrolase enzyme which could suppress cellular dNTP pools to inhibit retrovirus reverse transcription. Vpx is restricted to viruses of the human immunodeficiency virus type 2 (HIV-2), simian immunodeficiency virus (SIV) of sooty mangabey (SIVsm), SIV of red-capped mangabey (SIVrcm), and SIV of macaque (SIVmac) lineages and is absent from HIV-1. In some other primate lentivirus lineages, a different accessory protein, viral protein R (Vpr), appears to carry out this function of degrading SAMHD1. These include, e.g., SIVmus (SIV infecting mustached monkeys), SIVdeb (SIV infecting De Brazza's monkeys) and SIVagmVer (SIV infecting Vervet African green monkey), which all encode a Vpr protein with broad specificity against primate SAMHD1 proteins (see, e.g., Lim et al., Cell Host Microbe. 11: 194-204, 2012).

Any of these SAMHD1-inhibiting viral accessory proteins can be employed in the practice of the present invention. Vpx and Vpr proteins from various retroviruses that are capable of degrading SAMHD1 have been reported and characterized in the art. See, e.g., Tristem et al., EMBO J. 11:3405-12, 1992; Yu et al., J. Virol. 65:5088-5091, 1991; Kappes et al., Virology 184:197-209, 1991; Goujon et al., J. Virol. 82:12335-45, 2008; Belshan et al., Virology 346118-126, 2006; Goujon et al., Retrovirology 42, 2007; and Park et al., J. Acquir. Immune Defic. Syndr. Hum. Retrovirol. 8:335-344, 1995. Nucleotide and amino acid sequences of these proteins are also known. In addition, mechanism and structural requirement for Vpx or Vpr to degrade SAMHHD1 have been delineated in the art. For example, it has been reported that the amino terminus of Vpx contains an activation domain that serves as the binding site for a cellular restriction factor. See, e.g., Gramberg et al., J. Virol. 84:1387-96, 2010; Bergamaschi et al., J Virol. 83:4854-4860, 2009; Sharova et al., PLoS Pathog. 4:e1000057, 2008; Lim et al., Cell Host Microbe. 11: 194-204, 2012; and Wei et al., Cell Microbiol. 14:1745-56, 2012.

Based on Vpx/Vpr structural and functional information known in the art, recombinant production and purification of Vpx or Vpr proteins or functional fragments can be readily carried out via standard techniques of molecular biology. Expression and packaging of a Vpx or Vpr protein along with a viral vector (e.g., a lentiviral vector) into a virion (e.g., an HIV-1 virion) can also be performed in accordance with methods well known and routinely practiced in the art or the specific protocols exemplified herein. See, e.g., Goujon et al., Gene Ther. 13:991-4, 2006; Gramberg et al., J. Virol. 84:1387-96, 2010; Hofmann et al., J. Virol. 86:12552-60, 2012; Swan et al., Gene Ther. 13:1480-92, 2006; Ayinde et al., Retrovirology 7:35, 2010; and Sharova et al., PLoS Pathog. 4:e1000057, 2008. Other types of SAMHD1 inhibitors, e.g., small molecule organic compounds, can also be obtained via routinely practiced methods of organic chemistry and biochemistry.

Based on Vpx/Vpr structural and functional information known in the art, recombinant production and purification of Vpx or Vpr proteins or functional fragments can be readily carried out via standard techniques of molecular biology. Expression and packaging of a Vpx or Vpr protein along with a viral vector (e.g., a lentiviral vector) into a virion (e.g., an HIV-1 virion) can also be performed in accordance with methods well known and routinely practiced in the art or the specific protocols exemplified herein. See, e.g., Goujon et al., Gene Ther. 13:991-4, 2006; Gramberg et al., J. Virol. 84:1387-96, 2010; Hofmann et al., J. Virol. 86:12552-60, 2012; Swan et al., Gene Ther. 13:1480-92, 2006; Ayinde et al., Retrovirology 7:35, 2010; and Sharova et al., PLoS Pathog. 4:e1000057, 2008. Other types of SAMHD1 inhibitors, e.g., small molecule organic compounds, can also be obtained via routinely practiced methods of organic chemistry and biochemistry.

IV. Inhibitors of mTOR Complexes Suitable for the Invention

Some aspects of the present invention relate to novel methods and compositions for high frequency targeting and efficient payload delivery of viral vectors to host cells. The present inventors discovered that a concurrent inhibition of restriction factor SAMHD1 and mTOR complexes in host cells allows for more efficient viral transduction into the host cell. “Inhibitors of mTOR complexes” (or “mTOR complex inhibitors”) suitable for the invention are any compounds that inhibit or antagonize one or both of the mTOR complexes, mTORC1 and/or mTORC2. These include compounds that inhibit the mTOR kinase, as well as compounds that otherwise suppress or antagonize signaling activities of the mTOR complexes or negatively affect their biological properties (e.g., destabilizing or disrupting the protein complexes). For example, they can be compounds that do not directly impact the mTOR kinase, but through other components of the mTOR protein complexes (e.g., Raptor or RICTOR) can disrupt, or inhibit the formation of, the mTORC1 complex and/or the mTORC2 complex or inhibit interaction of the complexes with downstream signaling molecules.

In some embodiments of the invention, the employed inhibitor is a compound that antagonizes the mTOR kinase (mTOR inhibitors). Various mTOR inhibitors known in the art can be employed in the practice of particular embodiments of the invention. As used herein, the term “mTOR inhibitor” or “mTOR inhibitor compound” broadly encompasses any compounds that directly or indirectly inhibit or antagonize mTOR biological activities (e.g., kinase activity) or mTOR mediated signaling activities. Thus, the mTOR inhibitor can be a compound that suppresses mTOR expression or affects its cellular stability, a compound that inhibits or prevents formation of mTOR complexes, a compound that inhibits mTOR binding to its intracellular receptor FKBP12, a compound that inhibits or antagonizes enzymatic activities of mTOR, or a compound that otherwise inhibits mTOR interaction with downstream molecules.

Some embodiments of the invention employ rapamycin. Rapamycin (Vezina et al., J. Antibiot. 1975; 28: 721\u20136), also known as Sirolimus, is an immunosuppressant drug used to prevent rejection in organ transplantation. It prevents activation of T cells and B-cells by inhibiting their response to interleukin-2 (IL-2). It was approved by the FDA in September 1999 and is marketed under the trade name Rapamune by Pfizer. Rapamycin is an allosteric mTOR inhibitor. Other than rapamycin, any compounds that specifically mimic or enhance the biological activity of rapamycin (e.g., binding to the FKBP12-rapamycin-binding domain of mTOR and/or inhibiting mTOR kinase activity) can be used in the invention. For example, mTOR is the principal cellular target of rapamycin. Thus, rapamycin analogs or functional derivatives with similar or improved inhibitory activity on mTOR may be suitable for particular embodiments of the present invention. These include rapamycin analog compounds known in the art. Examples include compounds described in, e.g., Ritacco et al., Appl Environ Microbiol. 2005; 71: 1971-1976; Bayle et al., Chemistry & Biology 2006; 13: 99-107; Wagner et al., Bioorg Med Chem Lett. 2005; 15:5340-3; Graziani et al., Org Lett. 2003; 5:2385-8; Ruan et al., Proc. Natl. Acad. Sci. USA 2008; 105:33-8; U.S. Pat. No. 5,138,051; and WO/2009/131631. Several semi-synthetic rapamycin analogs (also known as rapalogues) have been evaluated by pharmaceutical companies for clinical development, e.g., temsirolimus (CCI-779, Torisel, Wyeth Pharmaceuticals), everolimus (RAD001, Afinitor, Novartis Pharmaceuticals), and ridaforolimus (AP23573; formerly deforolimus, ARIAD Pharmaceuticals).

Some other embodiments of the invention can employ ATP-competitive mTOR inhibitors. These mTOR inhibitors are ATP analogues that inhibit mTOR kinase activity by competing with ATP for binding to the kinase domain in mTOR. Unlike rapamycin, which primarily inhibits only mTORC1, the ATP analogues inhibit both mTORC1 and mTORC2. Because of the similarity between the kinase domains of mTOR and the PI3Ks, mTOR inhibition by some of these compounds overlaps with PI3K inhibition. Some of the ATP-competitive inhibitors are dual mTOR/PI3K inhibitors (which inhibit both kinases at similar effective concentrations). Examples of such inhibitors include PI103, PI540, PI620, NVP-BEZ235, GSK2126458, and XL765. These compounds are all well known in the art. See, e.g., Fan et al., Cancer Cell 9:341-349, 2006; Raynaud et al., Mol. Cancer Ther. 8:1725-1738, 2009; Maira et al., Mol. Cancer Ther. 7: 1851-63, 2008; Knight et al., ACS Med. Chem. Left., 1: 39-43, 2010; and Prasad et al., Neuro. Oncol. 13: 384-92, 2011. Some other ATP-competitive mTOR inhibitors are more selective for mTOR (pan-mTOR inhibitors) which have an IC50 for mTOR inhibition that is significantly lower than that for PI3K. These include, e.g., PP242, INK128, AZD8055, AZD2014, OSIO27, TORKi CC223; and Palomid 529. These compounds have also been structurally and functionally characterized in the art. See, e.g., Apsel et al., Nature Chem. Biol. 4: 691-9, 2008; Jessen et al., Mol. Cancer Ther. 8 (Suppl. 12), Abstr. B 148, 2009; Pike et al., Bioorg. Med. Chem. Lett. 23:1212-6, 2013; Bhagwat et al., Mol. Cancer Ther. 10:1394-406, 2011; and Xue et al., Cancer Res. 68: 9551-7, 2008.

Additional ATP-competitive mTOR inhibitors that can be employed in the present invention include, e.g., WAY600, WYE354, WYE687, and WYE125132. See, e.g., Yu et al., Cancer Res. 69: 6232-40, 2009; and Yu et al., Cancer Res. 70: 621-31, 2010. These compounds all have greater selectivity for mTORC1 and mTORC2 over PI3K. They are derived from WAY001, which is a lead compound identified from a high-throughput screen directed against recombinant mTOR and which is more potent against PI3K than against mTOR. Various other mTOR inhibitors known in the art can also be used in the practice of the present invention. These include, e.g., Torin 1 (Thoreen et al., J. Biol. Chem. 284: 8023-32, 2009), Torin2 (Liu et al., J. Med. Chem. 54:1473-80, 2011), Ku0063794 (Garcia-Martinez et al., Biochem. J. 421: 29-42, 2009), WJD008 (Li et al., J. Pharmacol. Exp. Ther. 334: 830-8, 2010), PKI402 (Mallon et al., Mol. Cancer Ther. 9: 976-84, 2010), NVP-BBD130 (Marone et al., Mol. Cancer Res. 7: 601-13, 2009), NVP-BAG956 (Marone et al., Mol. Cancer Res. 7: 601-13, 2009), and OXA-01 (Falcon et al., Cancer Res. 71: 1573-83, 2011).

Other than mTOR inhibitors that bind to and directly inhibit mTORC1 and/or mTORC2 complexes, compounds which antagonize mTOR activities in other manners may also be employed in the practice of the present invention. These include, e.g., Metformin which indirectly inhibits mTORC1 through activation of AMPK; compounds which are capable of targeted disruption of the multiprotein TOR complexes formed from mTORC1 and mTORC2, e.g., nutlin 3 and ABT-263 (Secchiero et al., Curr. Pharm. Des. 17, 569-77, 2011; and Tse et al., Cancer Res. 68: 3421-8, 2008); compounds which antagonize or inhibit phosphatidic acid mediated activation of mTORs, e.g., HTS-1 (Veverka et al., Oncogene 27: 585-95, 2008); and compounds which block the activity of mTORC1 activator RHEB, e.g., farnesylthiosalicylic acid (McMahon et al., Mol. Endocrinol. 19:175-83, 2005).

Suitable compounds for use in particular embodiments of the invention also include novel inhibitors of mTOR complexes or mTOR inhibitors (e.g., other rapamycin analogs) that can be identified in accordance with screening assays routinely practiced in the art. For example, a library of candidate compounds can be screened in vitro for mTOR inhibitors or rapamycin derivatives that inhibit mTOR. This can be performed using methods as described in, e.g., Yu et al., Cancer Res. 69: 6232-40, 2009; Livingstone et al., Chem Biol. 2009, 16:1240-9; Chen et al., ACS Chem Biol. 2012, 7:715-22; and Bhagwat et al., Assay Drug Dev Technol. 2009, 7:471-8. The candidate compounds can be randomly synthesized chemical compounds, peptide compounds or compounds of other chemical nature. The candidate compounds can also comprise molecules that are derived structurally from known mTOR inhibitors described herein (e.g., rapamycin or analogs).

The various inhibitors of mTOR complexes (e.g., mTOR inhibitors) described herein can be readily obtained from commercial sources. For example, rapamycin, some rapalogues described herein, and various ATP-competitive mTOR inhibitors (e.g., Torin 1) can be purchased from a number of commercial suppliers. These include, e.g., EMD Chemicals, R&D Systems, Sigma-Aldrich, MP Biomedicals, Enzo Life Sciences, Santa Cruz Biotech, and Invitrogen. Alternatively, the inhibitors of mTOR complexes can be generated by de novo synthesis based on teachings in the art via routinely practiced protocols of organic chemistry and biochemistry. For example, methods for synthesizing rapamycin are described in the art, e.g., Ley et al., Chemistry. 2009;15:2874-914; Nicolaou et al., J. Am. Chem. Soc. 1993, 115: 4419; Hayward et al., J. Am. Chem. Soc. 1993, 115: 9345; Romo et al., J. Am. Chem. Soc. 1993, 115: 7906; Smith et al., J. Am. Chem. Soc. 1995, 117: 5407-5408; and Maddess et al., Angew. Chem. Int. Ed. 2007, 46, 591. Structures and chemical synthesis of various other mTOR inhibitors suitable for the invention are also well characterized in the art.

V. Enhancing Viral Transduction by Co-Inhibiting SAMHD1 and mTOR Complexes

The invention further provides methods and compositions for enhanced viral transduction into the host cell that is either resting or pre-stimulated for differentiation. In some preferred embodiments, the host cells are unstimulated cells. Examples include non-stimulated stem cells (e.g., human CD34+ cells) or resting T cells (e.g., human CD4+ T cells). Some of the methods can be used to enhance transduction efficiency of recombinant retroviruses or retroviral vectors expressing various exogenous genes. For example, recombinant retroviruses expressing an exogenous gene or heterologous polynucleotide sequence can be transduced into host cells with enhanced transduction efficiency in various gene therapy and agricultural bioengineering applications. In some embodiments, the methods are intended for enhanced viral transduction in gene therapy. For example, a current problem with clinical stem cell based therapy is that viral vector entry and payload delivery does not occur without some form of stem cell proliferation. This potentially can result in differentiation of stem cells and loss of stem cell function when placed back into the host.

In some embodiments, methods of the invention involve transfecting a retroviral vector into a host cell (e.g., a stem cell such as human HSCs) in the presence of an SAMHD1 inhibitor and an inhibitor of mTOR complexes (e.g., rapamycin). The host cell can be contacted with the vector in vitro, in vivo (e.g., in a human or non-human subject), or ex vivo (in vitro transfected cells being reintroduced into a subject, e.g., via injection). The cells can be contacted with the viral vector and the two inhibitors in any order. Thus, the two inhibitor compounds can be contacted with the cell prior to, simultaneously with, or subsequent to addition of the retroviral vector or recombinant retrovirus. In addition, the two inhibitors themselves can be contacted with the cells in any desired order. For example, the target host cell can be treated with the mTOR inhibitor (e.g., rapamycin) prior to, simultaneously with, or subsequent to treatment with the SAMHD1 inhibitor and/or contacting with the viral vector or recombinant virus. In some embodiments, the target host cell is contacted concurrently with the SAMHD1 inhibitor and the viral vector. As exemplified herein, this can be achieved by conjugating the SAMHD1 inhibitor to the virion to be transduced. Regardless of the particular order by which the target cell is contacted, the treatment is followed by culturing the host cells under suitable conditions so that the viral vector or virus can be transduced into the cells.

Methods of the invention can be employed for enhancing transduction efficiency of various recombinant viruses or viral vectors used for gene transfer in many settings. In some embodiments, methods of the invention are used for promoting transduction of retroviruses or retroviral vectors, e.g., lentiviral vectors. Retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These elements contain strong promoter and enhancer sequences and are also required for integration in the host cell genome.

Retroviral vectors or recombinant retroviruses are widely employed in gene transfer in various therapeutic or industrial applications. For example, gene therapy procedures have been used to correct acquired and inherited genetic defects, and to treat cancer or viral infection in a number of contexts. The ability to express artificial genes in humans facilitates the prevention and/or cure of many important human diseases, including many diseases which are not amenable to treatment by other therapies. For a review of gene therapy procedures, see Anderson, Science 256:808-813, 1992; Nabel & Feigner, TIBTECH 11:211-217, 1993; Mitani & Caskey, TIBTECH 11:162-166, 1993; Mulligan, Science 926-932, 1993; Dillon, TIBTECH 11:167-175, 1993; Miller, Nature 357:455-460, 1992; Van Brunt, Biotechnology 6:1149-1154, 1998; Vigne, Restorative Neurology and Neuroscience 8:35-36, 1995; Kremer & Perricaudet, British Medical Bulletin 51:31-44, 1995; Haddada et al., in Current Topics in Microbiology and Immunology (Doerfler & Bohm eds., 1995); and Yu et al., Gene Therapy 1:13-26, 1994.

In order to construct a retroviral vector for gene transfer, a nucleic acid encoding a gene of interest is inserted into the viral genome in the place of certain viral sequences to produce a viral construct that is replication-defective. In order to produce virions, a producer host cell or packaging cell line is employed. The host cell usually expresses the gag, pol, and env genes but without the LTR and packaging components. In some embodiments, an SAMHD1-inhibiting accessory protein or polypeptide (Vpx or Vpr) may also be packaged into the virions as exemplified herein. When the recombinant viral vector containing the gene of interest together with the retroviral LTR and packaging sequences is introduced into this cell line (e.g., by calcium phosphate precipitation), the packaging sequences allow the RNA transcript of the recombinant vector to be packaged into viral particles, which are then secreted into the culture media. The media containing the recombinant retroviruses is then collected, optionally concentrated, and used for transducing host cells (e.g., stem cells) in gene transfer applications.

Suitable host or producer cells for producing recombinant retroviruses or retroviral vectors according to the invention are well known in the art (e.g., 293T cells exemplified herein). Many retroviruses have already been split into replication defective genomes and packaging components. For other retroviruses, vectors and corresponding packaging cell lines can be generated with methods routinely practiced in the art. The producer cell typically encodes the viral components not encoded by the vector genome such as the gag, pol and env proteins. The gag, pol and env genes may be introduced into the producer cell and stably integrated into the cell genome to create a packaging cell line. The retroviral vector genome is then introduced-into the packaging cell line by transfection or transduction to create a stable cell line that has all of the DNA sequences required to produce a retroviral vector particle. Another approach is to introduce the different DNA sequences that are required to produce a retroviral vector particle, e.g. the env coding sequence, the gag-pol coding sequence and the defective retroviral genome into the cell simultaneously by transient triple transfection. Alternatively, both the structural components and the vector genome can all be encoded by DNA stably integrated into a host cell genome.

The methods of the invention can be practiced with various retroviral vectors and packaging cell lines well known in the art. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. 66:2731-2739, 1992; Johann et al., J. Virol. 66:1635-1640, 1992; Sommerfelt et al., Virol. 176:58-59, 1990; Wilson et al., J. Virol. 63:2374-2378, 1989; Miller et al., J. Virol. 65:2220-2224, 1991; and PCT/US94/05700). Particularly suitable for the present invention are lentiviral vectors. Lentiviral vectors are retroviral vector that are able to transducer or infect non-dividing cells and typically produce high viral titers. Lentiviral vectors have been employed in gene therapy for a number of diseases. For example, hematopoietic gene therapies using lentiviral vectors or gamma retroviral vectors have been used for x-linked adrenoleukodystrophy and beta thalassaemia. See, e.g., Kohn et al., Clin. Immunol. 135:247-54, 2010; Cartier et al., Methods Enzymol. 507:187-198, 2012; and Cavazzana-Calvo et al., Nature 467:318-322, 2010. Methods of the invention can be readily applied in gene therapy or gene transfer with such vectors. In some other embodiments, other retroviral vectors can be used in the practice of the methods of the invention. These include, e.g., vectors based on human foamy virus (HFV) or other viruses in the Spumavirus genera.

In particular, a number of viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent. pLASN and MFG-S are examples are retroviral vectors that have been used in clinical trials (Dunbar et al., Blood 85:3048-305 (1995); Kohn et al., Nat. Med. 1:1017-102 (1995); Malech et al., Proc. Natl. Acad. Sci. U.S.A. 94:22 12133-12138 (1997)). PA317/pLASN was the first therapeutic vector used in a gene therapy trial. (Blaese et al., Science 270:475-480, 1995). Transduction efficiencies of 50% or greater have been observed for MFG-S packaged vectors (Ellem et al., Immunol Immunother. 44:10-20, 1997; Dranoff et al., Hum. Gene Ther. 1:111-2, 1997). Many producer cell line or packaging cell line for transfecting retroviral vectors and producing viral particles are also known in the art. The producer cell to be used in the invention needs not to be derived from the same species as that of the target cell (e.g., human target cell). Instead, producer or packaging cell lines suitable for the present invention include cell lines derived from human (e.g., HEK 292 cell), monkey (e.g., COS-1 cell), mouse (e.g., NIH 3T3 cell) or other species (e.g., canine). Some of the cell lines are disclosed in the Examples below. Additional examples of retroviral vectors and compatible packaging cell lines for producing recombinant retroviruses in gene transfers are reported in, e.g., Markowitz et al., Virol. 167:400-6, 1988; Meyers et al., Arch. Virol. 119:257-64, 1991 (for spleen necrosis virus (SNV)-based vectors such as vSNO21); Davis et al., Hum. Gene. Ther. 8:1459-67, 1997 (the “293-SPA” cell line); Povey et al., Blood 92:4080-9, 1998 (the “1MI-SCF” cell line); Bauer et al., Biol. Blood Marrow Transplant. 4:119-27, 1998 (canine packaging cell line “DA”); Gerin et al., Hum. Gene Ther. 10:1965-74, 1999; Sehgal et al., Gene Ther. 6:1084-91, 1999; Gerin et al., Biotechnol. Prog. 15:941-8, 1999; McTaggart et al., Biotechnol. Prog. 16:859-65, 2000; Reeves et al., Hum. Gene. Ther. 11:2093-103, 2000; Chan et al., Gene Ther. 8:697-703, 2001; Thaler et al., Mol. Ther. 4:273-9, 2001; Martinet et al., Eur. J. Surg. Oncol. 29:351-7, 2003; and Lemoine et al., I. Gene Med. 6:374-86, 2004. Any of these and other retroviral vectors and packaing producer cell lines can be used in the practice of the present invention.

Many of the retroviral vectors and packing cell lines used for gene transfer in the art can be obtained commercially. For example, a number of retroviral vectors and compatible packing cell lines are available from Clontech (Mountain View, Calif.). Examples of lentiviral based vectors include, e.g., pLVX-Puro, pLVX-IRES-Neo, pLVX-IRES-Hyg, and pLVX-IRES-Puro. Corresponding packaging cell lines are also available, e.g., Lenti-X 293T cell line. In addition to lentiviral based vectors and packaging system, other retroviral based vectors and packaging systems are also commercially available. These include MMLV based vectors pQCXIN, pQCXIQ and pQCXIH, and compatible producer cell lines such as HEK 293 based packaging cell lines GP2-293, EcoPack 2-293 and AmphoPack 293, as well as NIH/3T3-based packaging cell line RetroPack PT67. Any of these and other retroviral vectors and producer cell lines may be employed in the practice of the present invention.

Some embodiments of the invention relate to the transfer and recombinant expression of various exogenous genes or heterologous polynucleotide sequences. In some of these embodiments, the gene or heterologous polynucleotide sequence is derived from a source other than the retroviral genome which provides the backbone of the vector used in the gene transfer. The gene may be derived from a prokaryotic or eukaryotic source such as a bacterium, a virus, a yeast, a parasite, a plant, or an animal. The exogenous gene or heterologous polynucleotide sequence expressed by the recombinant retroviruses can also be derived from more than one source, i.e., a multigene construct or a fusion protein. In addition, the exogenous gene or heterologous polynucleotide sequence may also include a regulatory sequence which may be derived from one source and the gene from a different source. For any given gene to be transferred via the viral vectors, a recombinant retroviral vector can be readily constructed by inserting the gene operably into the vector, replicating the vector in an appropriate packaging cell as described above, obtaining viral particles produced therefrom, and then infecting target cells (e.g., stem cells) with the recombinant viruses.

In some embodiments, the exogenous gene or heterologous polynucleotide sequence harbored by the recombinant retrovirus is a therapeutic gene. The therapeutic gene can be transferred, for example to treat cancer cells, to express immunomodulatory genes to fight viral infections, or to replace a gene's function as a result of a genetic defect. The exogenous gene expressed by the recombinant retrovirus can also encode an antigen of interest for the production of antibodies. In some exemplary embodiments, the exogenous gene to be transferred with the methods of the present invention is a gene that encodes a therapeutic polypeptide. For example, transfection of tumor suppressor gene p53 into human breast cancer cell lines has led to restored growth suppression in the cells (Casey et al., Oncogene 6:1791-7, 1991). In some other embodiments, the exogenous gene to be transferred with methods of the present invention encodes an enzyme. For example, the gene can encode a cyclin-dependent kinase (CDK). It was shown that restoration of the function of a wild-type cyclin-dependent kinase, p16INK4, by transfection with a p16INK4-expressing vector reduced colony formation by some human cancer cell lines (Okamoto, Proc. Natl. Acad. Sci. U.S.A. 91:11045-9, 1994). Additional embodiments of the invention encompass transferring into target cells exogenous genes that encode cell adhesion molecules, other tumor suppressors such as p21 and BRCA2, inducers of apoptosis such as Bax and Bak, other enzymes such as cytosine deaminases and thymidine kinases, hormones such as growth hormone and insulin, and interleukins and cytokines.

The recombinant retroviruses or retroviral vectors expressing an exogenous gene can be transduced into any target cells in the presence of an inhibitor of mTOR complexes (e.g., an mTOR inhibitor such as an ATP-competitive inhibitor or allosteric inhibitor rapamycin) for recombinant expression of the exogenous gene. As exemplified herein, preferred target cells for the present invention are stem cells. Stem cells suitable for practicing the invention include and are not limited to hematopoietic stem cells (HSC), embryonic stem cells or mesenchymal stem cells. They include stem cells obtained from both human and non-human animals including vertebrates and mammals. Other specific examples of target cells include cells that originate from bovine, ovine, porcine, canine, feline, avian, bony and cartilaginous fish, rodents including mice and rats, primates including human and monkeys, as well as other animals such as ferrets, sheep, rabbits and guinea pigs.

Transducing a recombinant retroviral vector into the target cell in the presence of an inhibitor of mTOR complexes (e.g., rapamycin) and/or an SAMHD1 inhibitor can be carried out in accordance with protocols well known in the art or that exemplified in the Examples below. For example, the host cell (e.g., HSCs) may be pre-treated with the inhibitor compound prior to transfection with the retroviral vector. Alternatively, the target host cell can be transfected with the viral vector in the presence of an inhibitor of mTOR complexes described herein (e.g., rapamycin or an analog compound). The concentration of the inhibitor to be used can be easily determined and optimized by the skilled artisans, depending on the nature of the compound, the recombinant vector or virus used, as well as when the cell is contacted with the compound (prior to or simultaneously with transfection with the vector). Typically, the inhibitor (rapamycin or an analog) should present in a range from about 10 nM to about 2 mM. Preferably, the compound used in the methods is at a concentration of from about 50 nM to about 500 μM, from about 100 nM to 100 μM, or from about 0.5 μM to about 50 μM. More preferably, the compound is contacted with the producer cell at a concentration of from about 1 μM to about 20 μM, e.g., 1 μM, 2 μM, 5 μM or 10 μM.

The invention also provides pharmaceutical combinations, e.g. kits, that can be employed to carry out the various methods disclosed herein. Such pharmaceutical combinations typically contain an SAMHD1 inhibitor (e.g., a Vpx or Vpr protein, or a functional derivative or fragment thereof) or a polynucleotide encoding the SAMHD1 inhibitor, an mTOR inhibitor compound (e.g., rapamycin or a rapamycin analog described herein), in free form or in a composition with one or more inactive agents, and other components. The pharmaceutical combinations can also contain one or more appropriate retroviral vectors (e.g., a lentiviral vector described herein) for cloning a target gene of interest. The pharmaceutical combinations can additionally contain a packaging or producer cell line (e.g., 293T cell line) for producing a recombinant retroviral vector that expresses an inserted target gene or polynucleotide of interest. Additional reagents can be provided in the pharmaceutical combinations or kits for packaging the SAMHD1 inhibitor along with the viral vector into virions. In some embodiments, the pharmaceutical combinations contain a host cell or target cell into which an exogenous gene harbored by the recombinant retroviral vector or virus is to be delivered. In various embodiments, the pharmaceutical combinations or kits of the invention can optionally further contain instructions or an instruction sheet detailing how to use the inhibitor of mTOR complexes (e.g., mTOR inhibitor such as rapamycin) to transduce recombinant retroviruses or retroviral vectors with enhanced efficiency.

### EXAMPLES

The following examples are provided to further illustrate the invention but not to limit its scope.

### Example 1

### Combination of Rapamycin and Vpx Enhances Lentiviral Transduction of Non-Stimulated CD34+ Cells

This Example describes effect of rapamycin or Vpx on lentiviral vector transduction efficacy of non-cytokine stimulated CD34+ cells. Preparation of HIV-1 virion containing an HIV-2 Vpx protein was carried out using the protocol described in Swan et al., Gene Ther. 13:1480-92, 2006 with some modifications. Specifically, an FG12 transfer vector (Qin et al., Proc. Natl. Acad. Sci. USA, 100:183-8, 2003) is used in place of CAD used in Swan et al., and plasmid pCG-239-Vpx (obtained from Dr. Jacek Skowronski) was included in the transfection mixture. As control, HIV-1 virion not containing the Vpx accessory protein was also prepared.

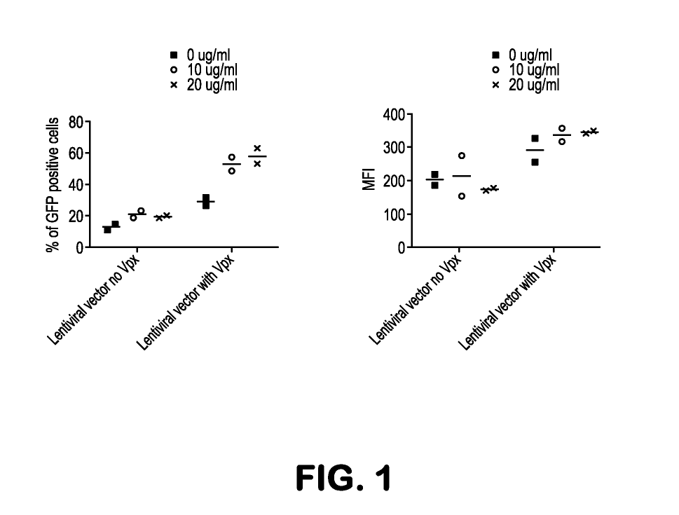

Lentiviral transduction of the cells were examined with no rapamycin or Vpx, rapamycin only, Vpx only, or both rapamycin and Vpx. As shown in FIG. 1, the results indicate that rapamycin or Vpx only increases transduction slightly over no treatment with either condition (FIG. 1, left panel). However, when both rapamycin and Vpx are present, transduction efficacy increased by 2.5-3 fold. Moreover, when the mean fluorescent intensity was evaluated from CD34+ cells treated with both rapamycin and Vpx it is apparent that GFP levels were increased over treatment with only rapamycin. Importantly, the results showed that combination of both rapamycin and Vpx enhances CD34+ cell lentiviral vector transduction greater than either alone or that would be predicted if each affect were simply additive. These findings indicate that the combination treatment with both rapamycin and Vpx increases transduction frequency of lentiviral vector virions into CD34+ cell in a syngergistic manner. This is consistent with increased vector copies per cell, based on the mean fluorescent intensity (MFI, right panel) results.

### Example 2

### Rapamycin/Vpx Enhance Lentiviral Vector Transduction of Resting T Cells

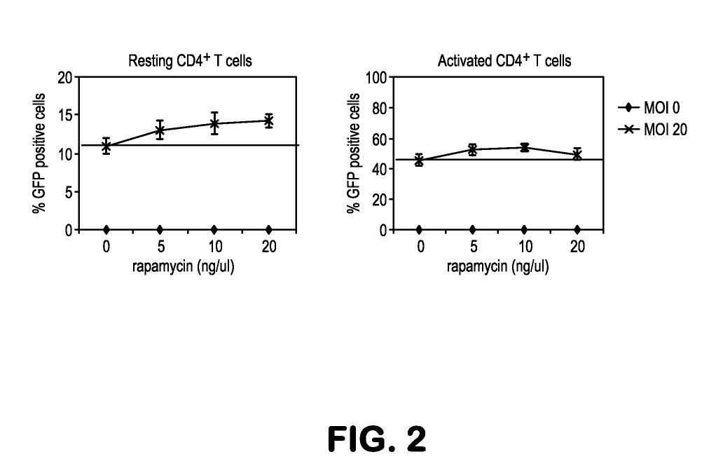

We also evaluated whether rapamycin would enhance lentiviral vector transduction of unstimulated CD4+ T cells. Specifically, freshly isolated human peripheral blood CD4+ CD25low, CD69low T cells or CD4+ T cells were not or cultured with various concentration of rapamycin for 12 hours and during that time exposed to no lentiviral vector (MOI 0) or lentiviral vector at 20 multiplicity of infection (MOI 20). The results are shown in FIG. 2. Shown in the left panel of FIG. 2 are CD4 T cells freshly isolated, non-stimulated (no cytokines or T cell mediated-activation), which were treated with lentiviral vector and rapamycin immediately after isolation for 12 hours. The cells were washed, and then stimulated with beads-bound with CD3 and CD28 antibody in the presence of 100 U/ml IL-2. Eight days later the cells were evaluated for GFP expression. The right panel of FIG. 2 shows CD4 T cells that were stimulated with bead-bound CD3 and CD28 antibody in the presence of 100 U/ml IL-2 for 1 day. On the second day of activation, the cells were treated with lentiviral vector and rapamycin immediately for 12 hours. The cells were then washed, and restimulated with beads-bound with CD3 and CD28 antibody in the presence of 100 U/ml IL-2. Six days later the cells were evaluated for GFP expression.

As shown in FIG. 2, rapamycin enhanced lentiviral vector transduction of unstimulated CD4+ T cells, but not activated CD4+ T cells. Moreover, increasing concentrations of rapamycin enhanced lentiviral vector transduction. The enhancement of lentiviral vector transduction of unstimulated, resting CD+4 T cells utilizing rapamycin is reminiscent of the limited increase in HIV infection found in the presence of Vpx. By functional extension of our findings with unstimulated CD34+ cells (shown in FIG. 1), rapamycin when used in combination with Vpx should also provide a synergistic increase in lentiviral transduction of resting, unstimulated CD4+ T cells.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

What is claimed is:

1. A method for enhancing transduction efficiency of a viral vector into a host cell, comprising transducing the host cell with the viral vector in the presence of (1) an mTOR inhibitor compound and (2) an inhibitor of SAM domain and HD domain-containing protein 1 (SAMHD1).

2. The method of claim 1, wherein the SAMHD1 inhibitor is packaged along with the vector into a virion prior to transducing the host cell.

3. The method of claim 1, wherein the host cell is not pre-stimulated with cytokine prior to transduction of the vector.

4. The method of claim 1, wherein the host cell is an unstimulated stem cell or a resting T cell.

5. The method of claim 4, wherein the stem cell is a hematopoietic stem cell (HSC),

6. The method of claim 1, wherein the viral vector is a lentiviral vector.

7. The method of claim 1, wherein the viral vector is a HIV-1 vector.

8. The method of claim 1, wherein the SAMHD1 inhibitor is accessory protein viral protein X (Vpx) or viral protein R (Vpr).

9. The method of claim 8, wherein the accessory protein Vpx is encoded by HIV-2, SIV_{SM}, or SIV_{MAC}.

10. The method of claim 8, wherein the accessory protein Vpr is encoded by SIVmus and SIVdeb.

11. The method of claim 1, wherein the mTOR inhibitor inhibits or antagonizes mTOR Complex 1 (mTORC1) and/or mTOR Complex 2 (mTORC2).

12. The method of claim 11, wherein the mTOR inhibitor is rapamycin or analog compound thereof.

13. The method of claim 1, wherein the vector is transduced into the stem cell at a multiplicity of infection (MOI) of 5, 10, 25, 50 or 100.

14. The method of claim 1, wherein the mTOR inhibitor compound is present during the entire transduction process or at specific intervals.

15. The method of claim 1, wherein the viral vector encodes a therapeutic agent.

16. The method of claim 1, wherein the viral vector is a non-integrating lentiviral vector.

17. A kit for delivering a therapeutic agent into a target cell with enhanced targeting frequency and payload delivery, comprising (a) a viral vector encoding the therapeutic agent, (b) an inhibitor of mTOR complexes, and (c) an SAMHD1 inhibitor or a polynucleotide encoding the SAMHD1 inhibitor.

18. The kit of claim 17, wherein the mTOR inhibitor is rapamycin or an analog thereof, and the SAMHD1 inhibitor is a Vpx or Vpr protein or functional fragment thereof.

19. The kit of claim 17, further comprising reagents for packaging the SAMHD1 inhibitor with the viral vector into a virion.

20. The kit of claim 17, wherein the viral vector is a lentiviral vector.
